(19) United States
(12) Patent Application Publication
(10) Pub. No.: US 2026/0256124 A1
(43) Pub. Date: Sep. 3, 2026

(54) MOSQUITO REARING AND PACKAGING

(71) Applicant: Senecio Ltd., Kfar-Saba (IL)

(72) Inventors: Hanan LEPEK, Kfar-Saba (IL); Ido NUSBAUM, Hod Hasharon (IL)

(73) Assignee: Senecio Ltd., Kfar-Saba (IL)

(21) Appl. No.: 18/836,808

(22) PCT Filed: Feb. 8, 2023
(86) PCT No.: PCT/IL2023/050138

§ 371 Date: Aug. 8, 2024

Related U.S. Application Data
(60) Provisional application No. 63/308,002, filed on Feb. 8, 2022.

(51) Int. Cl. A01K 67/362; A01K 67/366

(52) U.S. Cl. CPC A01K 67/362; A01K 67/366

(57) ABSTRACT

A device for rearing and sorting insects comprises a shaft, storage compartments, each compartment with a first closable opening for insertion of a water tray to hold pupae, and a second opening into said shaft, the inserted water tray being enclosed in the respective compartment apart from those two openings. A conveyor belt is aligned with an exit from the shaft to catch insects emerging from the pupae via the shaft. A classifier classifies insects on the conveyor belt into at least two classes; and a sorter sorts the insects on the conveyor based on output of the classifier to provide a sorted group of one of the classes.

### RELATED APPLICATION/S

This application claims the benefit of priority under 35 USC § 119(e) of U.S. Provisional Patent Application No. 63/308,002 filed Feb. 8, 2022, the contents of which are incorporated herein by reference in their entirety.

### FIELD AND BACKGROUND OF THE INVENTION

The present invention, in some embodiments thereof, relates to a mosquito rearing and packaging method and apparatus and, more particularly, but not exclusively, to such a method and apparatus using multi-level storage systems.

Mosquitoes are a major vector for different diseases, and one of the technologies developed in recent years involves releasing sterile male mosquitoes which, upon mating with wild mosquitoes, ensure that the female, who only mates once, is not fertilized, accordingly ensuring there will not be any offspring that will turn in to adults This can be achieved by releasing sterile males, or by releasing genetic modified mosquitoes, either males or females, which upon mating fail to breed.

However, these approaches, in order to be successful require the rearing of large numbers of mosquitoes, densities of sterile mosquitoes may need to approach numbers of 2,000-5,000 sterile mosquitoes per hectare in order to overwhelm the natural males and ensure that no fertile breeding occurs.

Some of the methods require separating males from females in order to release males only. Other methods require the separation in order to maintain a colony with males only and a colony with females only, after which, crossing between the two colonies, yields suitably modified mosquitoes who, when mating with wild counterpart sex mosquitoes, do not yield any offspring.

The rearing and separation processes are very involved and involve processing millions of insects. It is not practical to rear and separate insects of these volumes manually, nor would it be particularly safe for human operators. Also, a key important factor is the need of a low cost solution, to make the sterile insect technique widely accepted.

### SUMMARY OF THE INVENTION

The present embodiments may provide a device for rearing and sorting insects comprises a shaft, storage compartments, each compartment with a first closable opening for insertion of a water tray to hold pupae, and a second opening into said shaft, the inserted water tray being enclosed in the respective compartment apart from those two openings. A conveyor belt is aligned with an exit from the shaft to catch insects emerging from the pupae via the shaft. A classifier classifies insects on the conveyor belt into at least two classes; and a sorter sorts the insects on the conveyor based on output of the classifier to provide a sorted group of one of the classes.

The shaft provides a delivery passage for the insects from the trays to the conveyor, and the trays are placed around the shaft so that the shaft becomes a fully extendible delivery system. According to an aspect of some embodiments of the present invention there is provided a device for rearing and sorting insects comprising:
a shaft;
a plurality of storage compartments, each compartment configured with a first closable opening for insertion of a water tray to hold pupae, and a second opening into said shaft, the inserted water tray being enclosed by said respective compartment apart from said openings;
a conveyor mechanism aligned with an exit from said shaft to catch insects emerging from said pupae via said shaft;
a classifier configured to classify insects on said conveyor belt into at least two classes; and
a sorter for sorting insects on said conveyor based on output of said classifier to provide a sorted group of one of said classes.

Embodiments may comprise an air pressure source for moving air in said shaft to blow or suck said insects towards said conveyor. In an embodiment, said air pressure source is located below said conveyor, said conveyor having a surface being porous and said air pressure source being configured to suck insects down said shaft towards said conveyor surface and to immobilize said insects on said surface.

In an embodiment, said air pressure source is located at a top of said shaft to blow insects down said shaft onto said conveyor.

In an embodiment, said storage compartments are in a stack and said shaft extends upwardly for a height of said stack.

Embodiments may comprise an air pressure source located to blow insects in respective storage compartments out of a respective second opening.

In an embodiment, inner surfaces of said storage compartments are colored not to be black or brightly colored, thereby to discourage insects from landing thereon, and/or wherein light is provided in said shaft to encourage said insects to fly into said shaft.

In an embodiment, the sorter comprises at least one array of suction elements across a width of said conveyor.

In an embodiment, said classifier is configured to record a position across said width of an insect being classified, the device being configured to activate a suction element of said array, which suction element is at said recorded width position, if said insect being classified is classified for removal.

The device may be configured to measure a movement of said conveyor and to carry out said activation when said insect being classified reaches said array.

It is noted that the measurement need not be made each time but may be in the form of an occasional calibration. Embodiments may include a second array of suction elements for sucking up any insect not removed by said first array. These second suction elements may be designed for picking up the wanted insects and thus may use lower air speeds in order to be gentler to the insects. Also, as they are intended to suck up all the remaining insects they may be larger, to suck up multiple insects at the same time.

The device may comprise a cold chamber, the cold chamber connected to receive chilled air, the chilled air being at a temperature to immobilize said insects, the cold chamber being placed around said conveyor in association with cameras, thereby to image said insects in an immobilized state for said classifier. In an embodiment, said shaft is connected via a pipe to said conveyor. In an embodiment, the shaft is directly over said conveyor and said storage compartments are stacked on either side of said shaft, said shaft extending at least over a height of said storage compartments.

The device may include an array of at least two stacks of storage compartments on each side of said shaft. The stacks may be vertical stacks around a vertical shaft.

The device may comprise air pressure adjustment openings within said stacks.

The stacks may be removable.

The device may include an outlet for the desired class of insect, the outlet being configured for attachment of a removable storage element for filling with the desired class of insect for later distribution. According to a second aspect of the present invention there is a method for mass rearing and sorting of insects, comprising:
placing pupae in water trays;
placing said water trays in storage compartments in a stack opening into a shaft;
closing said storage compartment on all sides except into said shaft so that insects emerging from said pupae fly into said shaft;
providing air motion in said shaft to encourage said insects to fly towards a conveying mechanism;
classifying said insects on said conveying mechanism into at least two classes; and
sorting insects on said conveying mechanism based on output of said classifying to provide a sorted group of one of said classes.

According to a third aspect of the present invention there is provided a device for rearing and sorting insects comprising:
a shaft;
a plurality of storage compartments, each compartment configured with a first closable opening for insertion of a water tray to hold pupae, and a second opening into said shaft, the inserted water tray being enclosed by said respective compartment apart from said openings;
a conveyor belt aligned with an exit from said shaft to catch insects emerging from said pupae via said shaft;
a classifier configured to classify objects on said conveyor belt into wanted insects and to provide a count of said wanted insects and
a sorter for sorting insects on said conveyor based on output of said classifier to provide a sorted group of one of said classes.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1-3 are three views of a device for rearing and sorting insects according to a first embodiment of the present invention;

FIGS. 4-6 are three views of an implementation of the device of FIGS. 1-3;

FIGS. 7A and 7B are two views of the device of FIG. 1 with a cooling system attached;

FIG. 8 is a view of insects being carried along a conveyor belt according to an embodiment of the present invention;

FIG. 9 is a graph of arrival times of insects to the imaging position using embodiments of the present invention;

FIGS. 10A and 10B are views of suction devices for removal of insects from the conveyor belt according to embodiments of the present invention;

FIG. 11 is a cross-sectional view of a suction device below the conveyor belt;

FIG. 12 is a simplified view of rails for placement of racks according to the present invention;

FIGS. 13 and 14 are two views of an array of racks arranged around an extended shaft according to an embodiment of the present invention;

FIG. 15 is a simplified diagram illustrating an air pipe for use as a pressure source to provide suction according to embodiments of the present invention;

FIG. 16 is a simplified diagram illustrating rear rack doors with nozzles for providing pulses of air per individual rack or groups of racks, according to an embodiment of the present invention;

FIG. 17 is a simplified diagram illustrating a housing for racks according to an embodiment of the present invention, wherein the housing is able to enclose the rack and prevent insects from escaping in unwanted directions; and

FIG. 18 is a simplified diagram showing housings for insertion of racks and also a cold chamber arranged over the conveyor.

### DESCRIPTION OF SPECIFIC EMBODIMENTS OF THE INVENTION

The present invention, in some embodiments thereof, relates to a mosquito rearing and packaging method and apparatus and, more particularly, but not exclusively, to such a method and apparatus using multi-level storage systems.

The present embodiments comprise a solution which allows rearing millions of mosquitoes and also allows the separation of the males and females of all those millions of mosquitoes. The present embodiments may provide not only a highly automated process, but also a relatively efficient and accordingly low cost system.

Embodiments may provide an apparatus comprising the following elements, a multi level storage compartment and a shaft, a conveying system, a set of suction units to remove one designated class of mosquitoes, for example: females, and a set of suction units to remove a second designated class of mosquitoes, for example: males.

The shaft provides a fully extendible delivery system for getting the insects from the trays to the conveyor. Any number of trays may be added to the system simply by making the shaft a little longer or a little taller.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

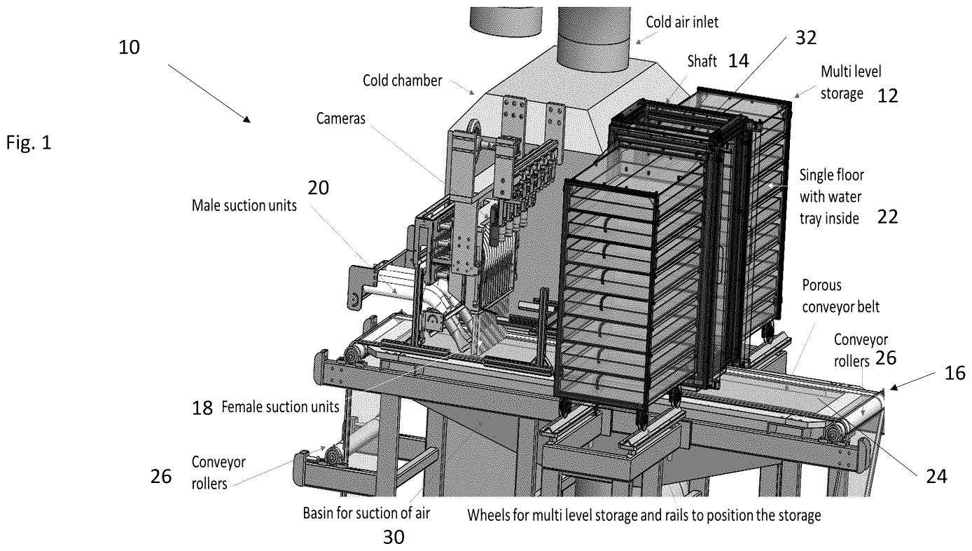

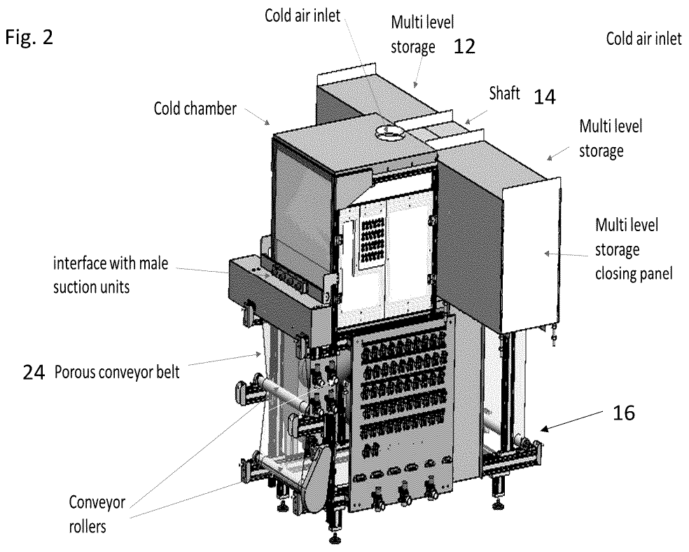

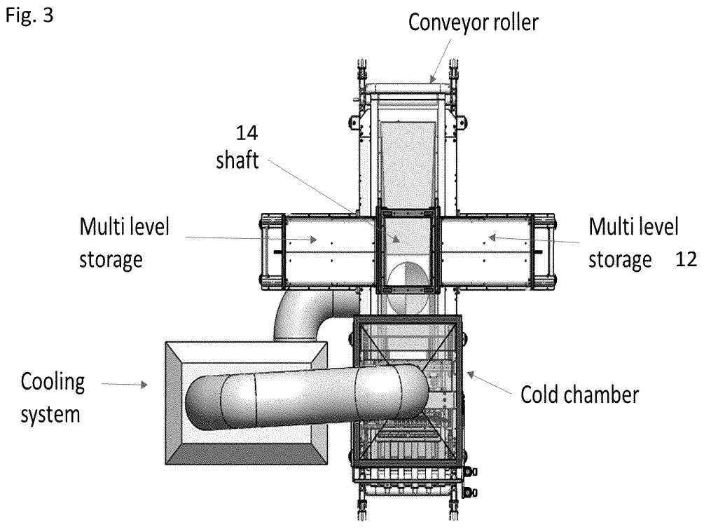

Referring now to the drawings, FIG. 1 illustrates a cross section of a device 10 for rearing and sorting mosquitoes according to an embodiment of the present invention with those different components: a multi level storage compartment 12, and a shaft 14, a conveying system 16, a set of suction units 18 to remove one designated class of mosquitoes, for example: females, and a set of suction units 20 to remove a second designated class of mosquitoes, for example: males. FIG. 2 is an external perspective view of the same device 10 and FIG. 3 is a view from the top.

The multi level storage compartment 12 comprises a set of floors. In each floor 22 there is located at least one water tray. Hence, the water trays are located one above the other. The multi level storage compartment 12 is the structure that holds the floors with trays together, and more specifically may provide a frame into which each tray can be pushed into each of the floors. The trays can be either pushed into a specific floor, with water already inside them, or they can be empty, and once they are in position, then they may be filled with water. The multi level storage unit structure may include at least two floors.

Each floor may have two openings, typically on different respective sides, for example on two oppositely facing sides. One side serves to push inside the water tray. The other side serves as the outlet for the adult mosquitoes to fly out. Each floor may have a roof, or a covering, leaving the mosquitoes no choice but to fly out of one of the two openings.

A suitable water level inside each of the water trays is about 2-4 cm height.

It is preferred that when the system is in operation, the water in each of the trays is heated to about 28 degree Celsius.

Heating the water can be achieved by placing a heating plate below the water tray, the water tray being manufactured from a material that may easily transfer heat, such as aluminium. It may also be made of other materials.

Additionally or alternatively, the surrounding air around the system and/or around the water trays may be heated. Thus air conditioning may be used instead of or additionally to heating the water.

The frame that holds the water trays may be positioned perpendicular to a conveying system such as conveying system 16 which includes a conveyor belt 24 and rollers 26.

Inside the water trays there are located mosquito pupae.

The pupa may generally be placed in each of the water trays before the system start operating. Furthermore, it is possible to fill only some of the trays with pupae, and fill in some others at a different timing, potentially to manipulate and affect the number of emerging mosquitoes over time that arrive towards the conveying system.

The surface of conveyor belt 24 may be porous.

The frame structure for the multi-level storage 12 is positioned adjacent to and above the conveyor belt 24.

The frame structure of the multi level storage 12 has two openings. When in operation, and when it is expected that the pupa inside the water trays start emerging and flying as adult mosquitoes, the opening away from the conveyor is closed, preventing the adult mosquitoes from flying out and away from the frame in the direction opposite to the conveyor.

While it is possible to have a different closing per each floor, it is easier to have one opening for the whole rear side of the multi level storage unit 12.

When in operation, the rear side opening may be closed and opening on the side not being the rear side, is open, allowing mosquitoes that emerge from the pupa, to fly out of the drawer they are located in. Each of the floors may be in the form of a drawer, and the water tray fits sliding onto the floor. Thus the multi-level storage unit resembles a chest of drawers.

The drawer may in embodiments be an operational drawer that can go in and out, for example on rails, or it may be only an empty space to allow entering the water trays to be slidingly inserted.

To summarise, pupa are placed inside the water in the water trays, and the water is held at about 28 degree Celsius, encouraging the pupae to start emerging into adult mosquitoes.

The width of each of the water trays may exactly fill the width of the drawer (the spacing of each floor), or may be narrower. It is preferred, in order to maximize the number of potential pupa, that the water tray is as close as practical to the width of the drawer.

In embodiments, the color of the frame from the inside, is chosen not to attract mosquitoes to stand on. Mosquitoes look for black or bright colors to choose for standing on and thus the frame is preferably not black or any bright color.

The frame may be positioned adjacent to the conveyor in such way a to form a guiding tunnel, herein the shaft 14, for the emerging mosquitoes so that they can fly from each of their drawers and towards the conveyor.

The shaft 14 may have a cross-sectional shape which is rounded, squared or any other shape.

In embodiments, an air flow is applied in order to cause the adult mosquitoes to fly towards the conveyor belt.

An air pressure source for providing such air flow may be a fan. The fan may be placed below the conveyor belt 24, for example in basin 30 and may suck air through the shaft down through the porous conveyor belt, thereby sucking the mosquitoes onto the conveyor belt.

In another embodiment, air flow is applied into each drawer from the rear side, that is the side away from the shaft and conveyor, to blow the insects towards the front, and causing adult mosquitoes which are flying inside each of the drawers to fly out and arrive at the conveyor belt. In an embodiment, insects emerge over time from each tray. In order to smooth the arrival rate of the insects onto the conveyor belt, it is possible to apply a pulse of air each time on a different drawer, so that insects from each tray are supplied separately to the belt. The pulses are applied to the different trays in succession to eject another set of insects each time. For example, at time 0, there is a pulse of air for 5 seconds across the water tray at level 1—highest, then there is a pause for 5 seconds, allowing the first batch of insects to fly towards the belt, giving a total time slot of 10 seconds. Other formats are applicable. For example, a 3 second pulse, followed by a wait of 1 second, and then a second pulse on the same tray. In this manner the first pulse may disturb the insects, causing them to fly within the boundaries of the tray, and the next pulse then pushes them out towards the shaft. In this manner, the adult insects continuously emerging from their pupa in the water, and then upon the next pulse, fly out towards the conveyor belt.

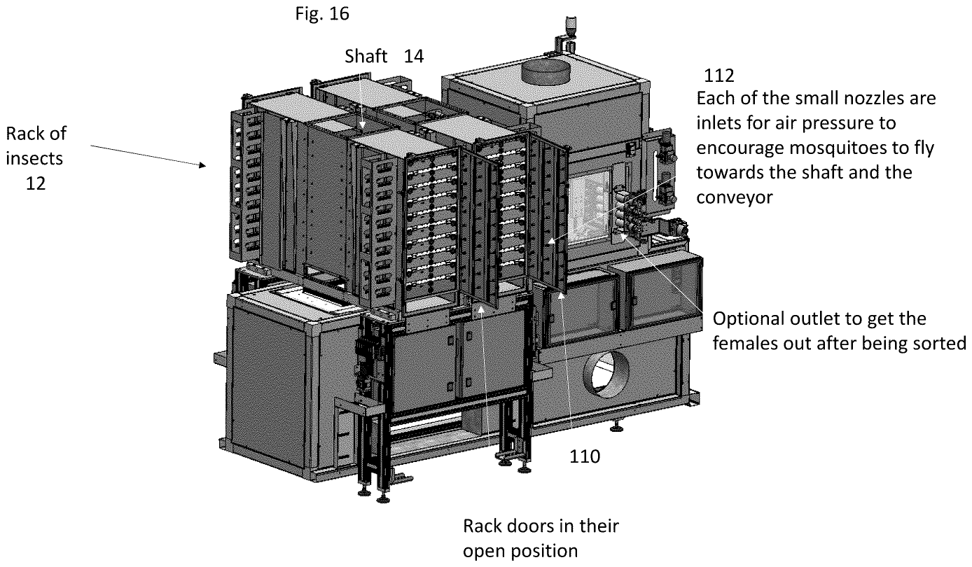

FIG. 16 below illustrates nozzles on the doors of each compartment that may be used to provide the pulses of air.

In another embodiment, a fan may be located at the top 32 of the shaft area, and may puff air down towards the conveyor belt 24, again to cause mosquitoes leaving the drawers to fly directly down to the conveyor belt 24.

It is furthermore possible to combine more than one option: having suction from below the conveyor belt, and having airflow from inside the each of the drawers, and/or having the suction below the conveyor belt and the fan from above, or all three.

It is possible to have openings within the frame structure in order to control the airflow, for example, having openings on the lower area of the frame, so that the suction through the conveyor belt may be high enough to immobilize the insects on the conveyor, while at the same time allowing low air speed within the frame, since the openings allow air flow

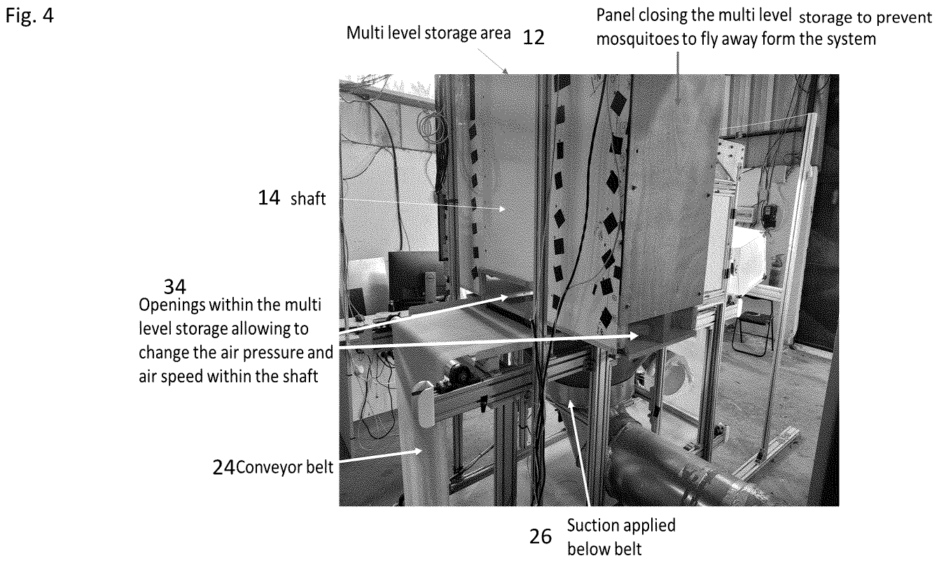

Such openings are shown in FIG. 4, to which reference is now made. Openings 34 are provided in the multi-level storage below the conveyor belt 24 to allow changes in air speed and pressure within shaft 14.

The multi level storage 12 may be positioned in such a way that it is at the edge of the conveyor, or in such position that it supports guiding of the flying mosquitoes towards a specific location, for example the middle of the conveyor belt.

The airflow inside the frame may be pulsated or continuous.

One or more lights may light the internal areas of the shaft to encourage adult mosquitoes to get out of the drawers as they are attracted to light. Additionally or alternatively, heat may be applied within the shaft or around the conveyor area where the shaft is located to attract the mosquitoes to get out from the drawers.

The light may also be pulsated, being switched between non-working and working states with different timing per each. Also, different lights may be applied, which when turned on and off using different timings, may create the effect of a moving shadow, which the mosquitoes inside the drawers may react to as a predator moving towards them, again causing them to fly out from the drawers and towards the conveyor belt.

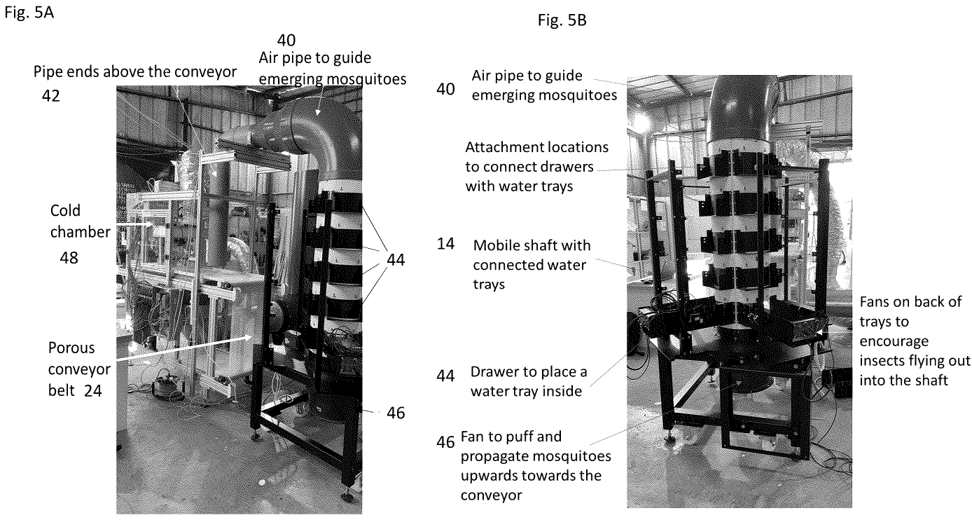

Reference is now made to FIGS. 5A and 5B, which show from different angles a further embodiment of the present invention. Instead of having the mosquitoes flying immediately downwards as they exit their drawers, a funnel or a pipe 40, having an ending 42 above the conveyor belt 24, may guide all the mosquitoes from all the floors in some direction towards the conveyor belt 24. A fan 46 may blow or suck the insects through the funnel, again guiding the flying mosquitoes to the conveyor belt.

The cross-sectional shape of the funnel or pipe 40 may be square, elliptic, circular or any other suitable shape.

In FIG. 5A, mosquitoes are located inside water trays inside black drawers 44. As they emerge they travel out of the drawer, and fan 46, located at the bottom of the shaft, puffs them upwards, through shaft 14 and pipe 40 and towards the conveyor 24. The shaft pipe ends at ending 42 above the conveyor and the mosquitoes fly towards and land on the conveyor 24. The conveyor then moves the mosquitoes towards an imaging location where cameras are combined with a cold chamber 48. The cold may immobilize the mosquitoes to a greater or lesser extent, allowing them to be photographed for image analysis to identify males and females. The image analysis may be used for separation and loading of the males.

The funnel or the pipe ending 42 may be positioned well above the conveyor 24, and mosquitoes flying out of ending 42 may fly on towards the conveyor. Alternatively, the ending 42 may be positioned very close, even touching the conveyor belt, for example with brushes.

As the mosquitoes arrive at the conveyor they are generally immobilized so they remain on the conveyor. The immobilization is not necessarily 100% and for example some mosquitoes may still be able to walk slowly on the conveyor. Immobilization at this point may be by suction from below the porous conveyor belt.

The conveyor continuously moves the mosquitoes arriving from the pipe or funnel 40 towards the imaging location which is near cold chamber 48. The cold chamber may allow a low temperature to be applied on the conveyor belt to improve the immobilization and increase the probability the mosquitoes will not move.

It is possible to lower or eliminate the suction below the conveyor and only use cold air, the cold air being applied or drawn along the conveyor belt from the cold chamber. Other methods for immobilising the mosquitoes may include the use of ammonia, CO₂ or any other suitable gas to anaesthetise the mosquitoes.

Applying cold air on the conveyor belt may achieved by having an enclosure area on top of the conveyor belt with two openings, one for the conveyor entrance and one for the conveyor exit at the opposite end, as well as an injection opening at the top to allow for injection of cold air. The cold air may be pushed into the enclosure through a mesh like structure to support a homogenous airflow, and an opening below the conveyor belt, may allow passage of air to the outside, for example back towards the entrance of the cold chamber 48.

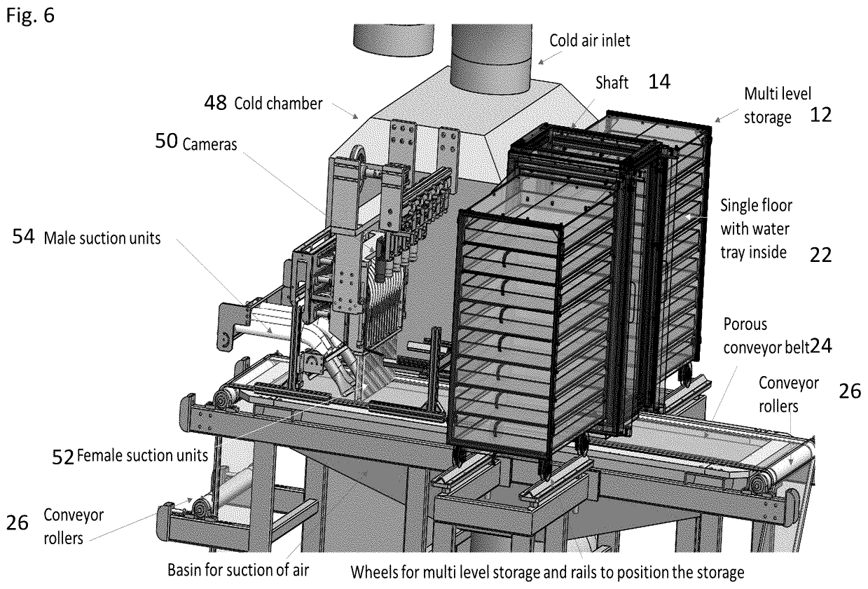

Referring now to FIG. 6, as the conveyor 24 moves insects to the imaging area under camera 50, the camera may take images of areas or stripes of the conveyor belt. It is possible that more than one camera is located above the conveyor, in order to cover the entire conveyor belt width.

The camera or cameras may be positioned to give suitable resolution so that the mosquito proboscis area is clearly seen, thus allowing a determination as to whether the currently imaged mosquito is a male or a female, the males having a more bushy shape.

For each image taken, computer software analyses the image, or the combination or set of images, and calculates the position of the object on the conveyor. It also decides if the object is such that it should be removed and considered as a female or a potential female, or it can be considered as a male.

At a certain distance away from the cameras are located two sets of suction elements 52, 54, positioned above the conveyor belt. Each suction element has an opening of about 1 cm diameter. When activated for a short time (e.g. 0.2 seconds) it sucks only what is below it on the conveyor belt if the suction is low enough.

Software calculates which single suction element is to be activated in order to suck any specific mosquito. One set is for sucking up wanted objects-say male mosquitoes, and one set is for sucking up unwanted objects, say female mosquitoes. The software determines which suction element to operate and when for each identified and classified object. Thus required suction is applied to suck the correct mosquito when the conveyor is moving.

There are several ways to obtain the desired result. In one embodiment, when the camera takes an image, the conveyor location is known. For example, an encoder indicates the current motor position. Image analysis decides whether the object is a wanted object or not, and then it is calculated which suction element, from which set, 52 or 54, needs to suck the mosquito. Is the current mosquito a potential female or a male? The distance between the XY coordinate of the mosquito in the image and the suction element is calculated. Then the conveyor position in order for the insect in question to be just below or near the relevant suction element is determined, and when that happens, a command is sent to trigger and activate the suction element.

The females are accordingly removed by having a fixed array 52 of suction elements, and the most suitable element is triggered with the correct timing. In an embodiment, in order to lower the possible female contamination rate within the males to be released, it is possible to trigger a few adjacent suction elements together and even to operate the elements for a longer time than needed to ensure the female is removed. This may be at the cost of removing potential male mosquitoes.

In another option, the same operation may use array 52 to remove males instead of removing females. For examples the females may be wanted for breeding.

After removal of the unwanted classification, the conveyor continues moving the remaining insects, typically now males only, towards the male loading area. At the male loading area, a second array 54, of male suction elements which do not harm the insects, suck the males from the conveyor belt and towards storage areas for later treatment. The male suction elements for example may use a lower air speed for suction.

The storage areas may be cartridges which are then taken to release devices such as aircraft, drones, land vehicles or even hand-held release devices for release anywhere from remote habitat to the city streets. The cartridges are opened, and the male mosquitoes that were loaded, are released into the environment.

The software which calculates the classification of the object, may additionally count how many objects of each class are on the conveyor. The data may then serve as a trigger to change the cartridge into which males are loaded with another empty cartridge.

The present embodiment, may obtain millions of male pupa on a small foot print machine yielding a very compact and low cost design system. In a single water tray of some 70 cm long and 40 cm wide, there may be some 50,000 pupae. Other may put different number, and using a 10 floor high multi-level storage some 500,000 pupae may be processed. Placing two multi level storage structures, one against the other, may provide for 1,000,000 pupae at once. In a space of 70×160 cm some 2 million mosquitoes can be reared.

As each mosquito emerges in its own time, the conveyor may move at a speed which prevents having too many of them at once on the conveyor belt. Too many insects close together may hinder accurate identification and classification. A suitable conveyor speed for such a number of insects may be between 1 cm/second and 6 cm/second.

For a conveyor with a 40 cm width, 5 cameras, with 12M pixel, each located such that the wider are of the camera sensor is about 8 cm, would allow good resolution imaging of the entire belt to identifying if a given mosquito is male or a female, for example by identifying whether it has a bushy antenna or not.

Accordingly, with 5 cameras, a moving conveyor, and two multi level storage units connected to a shaft, each unit holding 10 floors, and two additional units on the other side, some 2 million pupa mosquitoes can be processed.

In another embodiment, additional multi-level units may be connected one after the other in order to increase the overall number of water trays, which corresponds to a greater number of mosquito pupa

The following summarizes how the above system may work in one embodiment:
1 . Add water to the water trays
2 . Add mosquito pupa into the water in the water trays
3 . Place the water trays in a multi level storage device, holding the trays one above the other, each in a different drawer. The multi level storage device is positioned in such a way that one opening of the water trays is above the conveyor, and the other end is away from the conveyor.

The multi level storage unit may hold at least two water trays one above the other.
4. Water is heated within the trays to assist with hatching of the pupae.
5. The openings of the water trays opposite the conveyor location are closed, so that the mosquitoes are able to exit only in one direction, which is towards the conveyor belt.
6.The conveyor belt moves for the entire duration of pupa hatching within the system
7 . Mosquitoes are imaged on the conveyor belt at the imaging location and are classified into insects to be retained and insects to be removed.
8. A position is calculated for each insect that is required to be removed and when and which suction element is to be triggered to remove the specific object
9. Triggering of the suction units is based on the xy coordinates on the conveyor of the insect that needs to be removed
10. Removal by suction of the remaining, wanted, insects is carried out to load them into storage elements

The present embodiments may provide a packaging system for a specific class of insects in a large scale operation that provides millions of insects. One may alternatively make a system with a narrower conveyor belt, and/or fewer cameras, for smaller scale operation. Likewise one may have fewer trays in the stack, even just a single tray corresponding to a single drawer, with a cover over the drawer top, so as to let mosquitoes fly out only in one direction away from the drawer, and towards the conveyor belt.

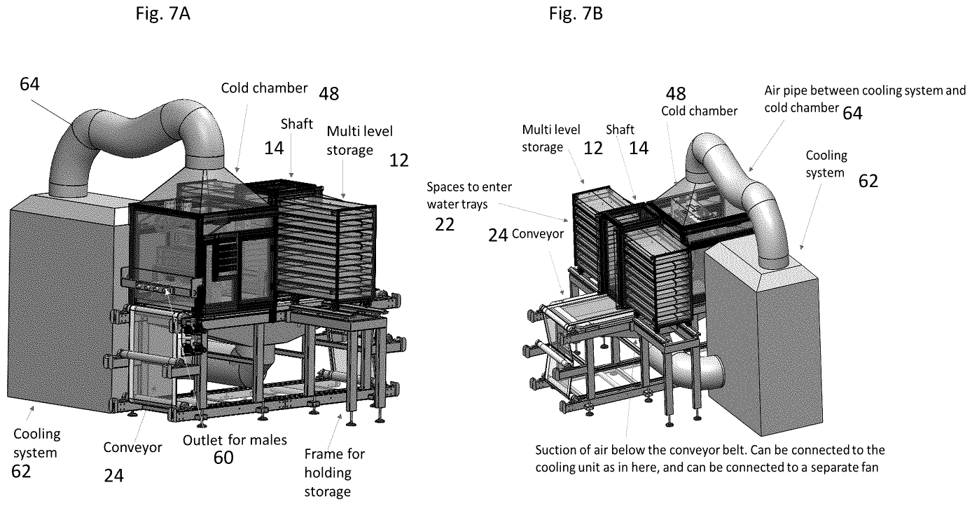

Reference is now made to FIGS. 7A and 7Bm which are two different views of a further embodiment of the present invention. Parts that are the same as in previous figures are given the same reference numerals and are not described again except as required for an understanding of the present embodiment.

The mosquito pupae are held inside the water trays, which are placed inside each of the floors 22 of the multi level storage. The water trays are heated. The mosquitoes emerge and fly towards the shaft 14 and are pulled down by the suction below the conveyor and arrive at the conveyor belt 24. The conveyor moves them into the region of cold chamber 48 where a temperature of around 7-9 degree Celsius is provided across the conveyor belt to render the insects stationary. The cold chamber 48 is provided with cold air from cooling system 62 via pipe 64. In another embodiment the mosquitoes may be kept stationary, that is in a non flying state, due to suction through the conveyor surface as discussed above. Some insects may still walk at 7 degrees, but not fly and the same applies with suction below the conveyor. In an embodiment suction is used until entry into the cold chamber, and from that point they the cold temperature is used.

As discussed, the conveyor moves under a set of cameras where images are taken and software decides upon the sex of the insect. In some embodiments classification may also relate to the species. The females are removed by the female suction units, and then the males are sucked via outlet for males 60 and packed into a storage unit.

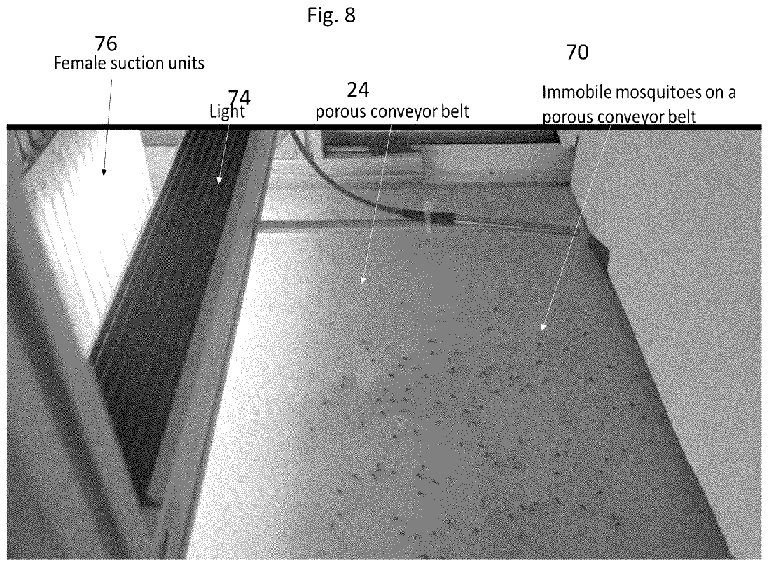

Reference is now made to FIG. 8, which is a simplified view from above of insects 70 on a porous conveyor belt 24. Lights in bank 74 illuminate the belt to help with imaging and female suction elements 76 are located across the line of travel to remove any insects designated for removal. The density of insects on the belt is relatively light to allow for clear identification and classification of the individual insects by the cameras.

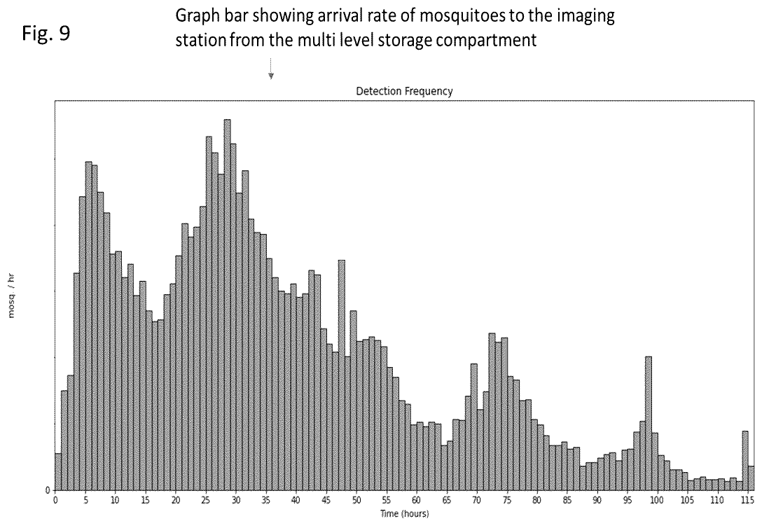

Reference is now made to FIG. 9, which is a qualitative graph showing arrival rate of mosquitoes over time. The mosquitoes emerge from pupae, mostly within the first 60 hours, in some two main waves, arrive at the shaft and then at the imaging location where the cameras are. In some species, the first wave may comprise mainly males, with the second wave comprising mainly females.

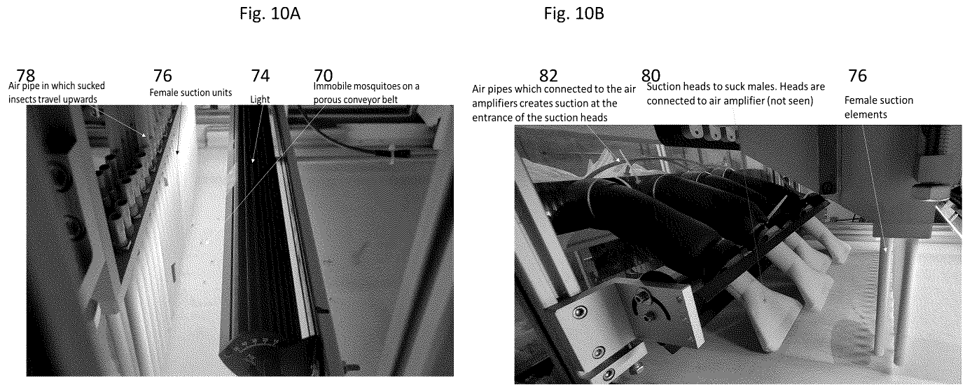

Referring now to FIG. 10A, mosquitoes 70 are seen on the conveyor belt 24 just before arriving at the female suction elements 76. Most, if not all, of the suction elements 76 are connected to air pipes 78. The elements extend across the width of the conveyor, so that for any x coordinate of an insect, there is an element above that can be switched on to suck up the insects. An advantage is that the female suction array is made up of fixed elements which do not move and are hence easier to maintain. They are located above the conveyor, and each time a specific suction unit is activated, for typically 0.2 seconds in order to suck the insect below.

For a continuous mode with high throughput it is preferred not to stop the conveyor, but rather to suck up the females as the conveyor moves. However, it is also possible to move the conveyor and operate in a pulse mode. That is, each time an insect to be removed arrives at the array, the conveyor is stopped. as the mosquito in question is below the suction element. As mentioned, it is also possible to activate more than one female suction unit at once.

Reference is now made to FIG. 10B, which shows male suction heads 80. The male heads are larger than the female suction elements 76 and are also connected to air pipes 82. The air pipes are connected to amplifiers to create suction at the suction heads. After the females are removed as described above, then all the remaining insects on the conveyor are considered as males. In order to expedite the operation of puffing the males and loading them into storage compartments, instead of using suction elements which preferably suck a single mosquito, large suction heads, are used. Preferably they are connected to air amplifiers, which safely allow the suction of the males away from the cold chamber and towards a receiving element such as for example a cartridge.

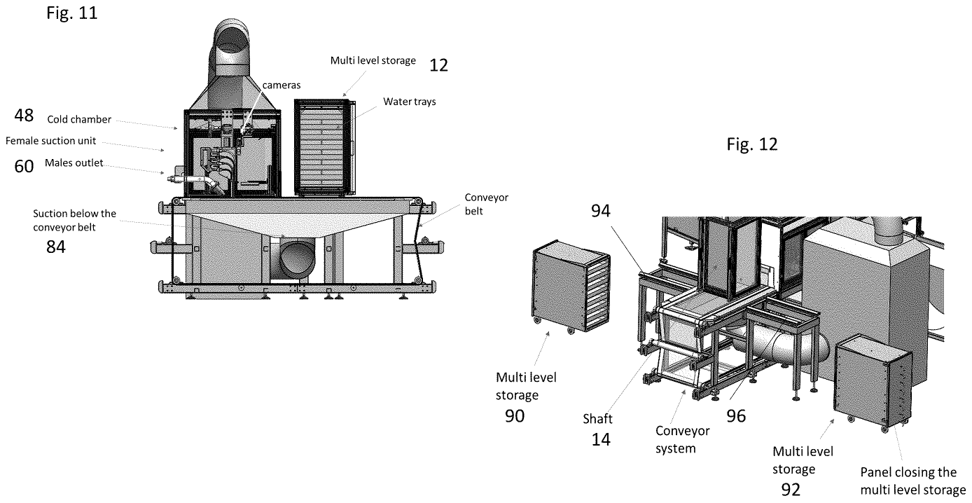

Reference is now made to FIG. 11 which is a cross section showing suction device 84 below the conveyor belt 24 and the multi level storage 12 located adjacent to the conveyor belt 24. The multi level storage 12 may be fixed, into which the water trays are loaded in and out, or water can also be directly filled and removed with valves. Alternatively, for ease of maintenance, the multi-level storage may be removable to be taken out of the structure holding the conveyor belt.

In the latter case, each time a different multi level storage is filled with new mosquito pupae, the multi-level storage is loaded onto the device and positioned so that the outlets are above the conveyor.

Reference is now made to FIG. 12, which shows two unloaded multi level storage units 90 and 92 which are standing next to the conveyor structure. The two storage units may be loaded onto rails 94 and 96 on either side of shaft 14 over the conveyor system.

Multi level storage unit 92 has a closing panel 98 on its right side, the side away from the shaft 14. During operation, the side which is facing away from the conveyor, is closed in order to force emerging mosquitoes to fly out only in one direction, towards the conveyor belt.

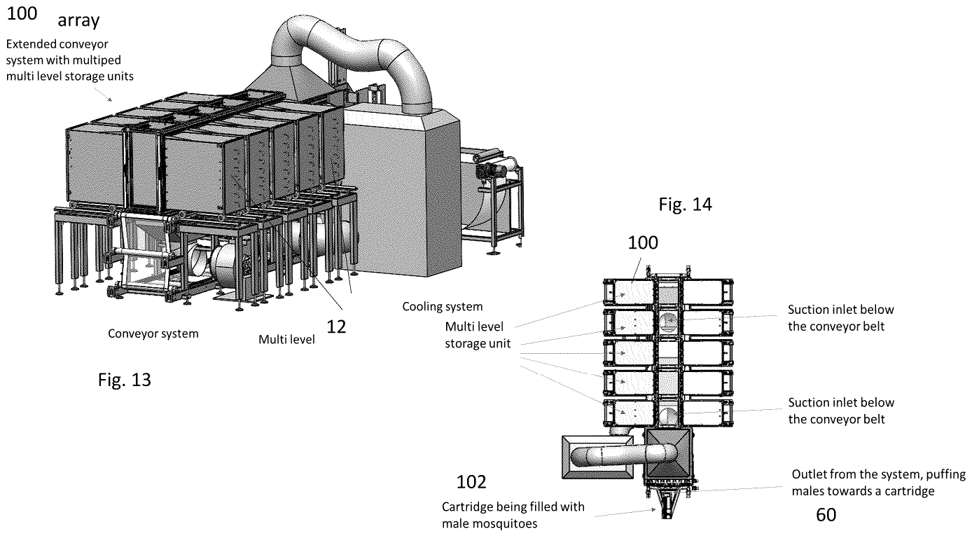

Reference is now made to FIG. 13, which illustrates an extended system with multiple multi level storage units 12 in an array 100. In such an embodiment, the conveyor 24 is longer in length to cover the full array of storage units and the larger numbers of insects in order to increase the total number of potential mosquitoes being processed and packed. FIG. 14 shows a top view of the embodiment of FIG. 13, additionally with a cartridge 102 at the exit, positioned to receive male mosquitoes being puffed out by the air amplifiers.

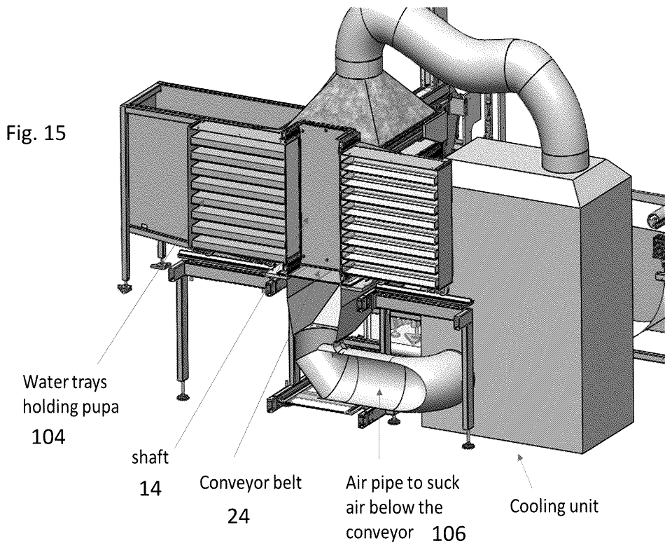

Reference is now made to FIG. 15, which is a cross section showing the water trays 104 and the shaft 14 with the suction below the conveyor belt 24 provided by an air pipe 106.

As discussed above, in any of the embodiments it is possible to change the mode of operation and have the female suction units to remove the males, and then the male suction head removes females across the conveyor belt and loads them into storage compartments, for later use, for example to provide breeding.

It is also possible to change the method of operation during work. For example during a first 24 hour period the machine may remove females and load males into cartridges, and the following 24 hours it may do the opposite.

Reference is now made to FIG. 16 which is a simplified diagram illustrating an array of four racks 12 around a single shaft 14. In FIG. 16, doors 110 for inserting the water trays are open, and nozzles 112 provide inlets for pulses of air to push the insects towards the shaft. Each tray may be pulsed in sequence or groups of trays may be pulsed together.

Nozzles 112 may be connected to a source of pressurized air. Differential sourcing may be provided per each nozzle or each tray or per groups of trays. Each time there is a pulse of air across one or a group of water trays, the pulse encourages or pushes insects to the shaft and to the moving belt. Judicious timing of the pulses among the different trays may support a better distribution over time of insects on the belt. To encourage the insects to fly out of the tray and towards the belt, it is possible to use attractants, or lures. One such lure may be the use of CO₂ at the location of the conveyor belt, attracting the insects to fly along the shaft and towards the conveyor belt. In other embodiments, instead of using air flow across the water trays to encourage the insects out of the trays and towards the belt, the use of a lure alone may be applied, although the use of air across the trays, or pulsated air is preferred dur to the ability to better control the process. In addition light may be applied in the shaft, encouraging the insects to fly towards the light.

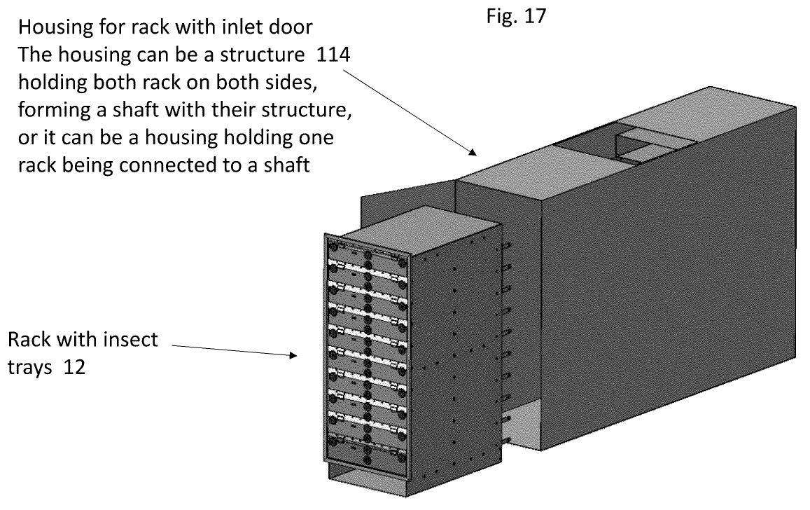

FIG. 17 illustrates a housing 114 for holding two racks on either side of a shaft. The housing may alternatively be designed for one rack only, or for larger numbers of racks. The housing compartment may incorporate a single or multiple heating units to provide controlled temperature for growing of the insects inside the rack. An additional closure may be provided on the opening on the side of the shaft which is kept closed until the insects begin to emerge, thus keeping the compartments warmer.

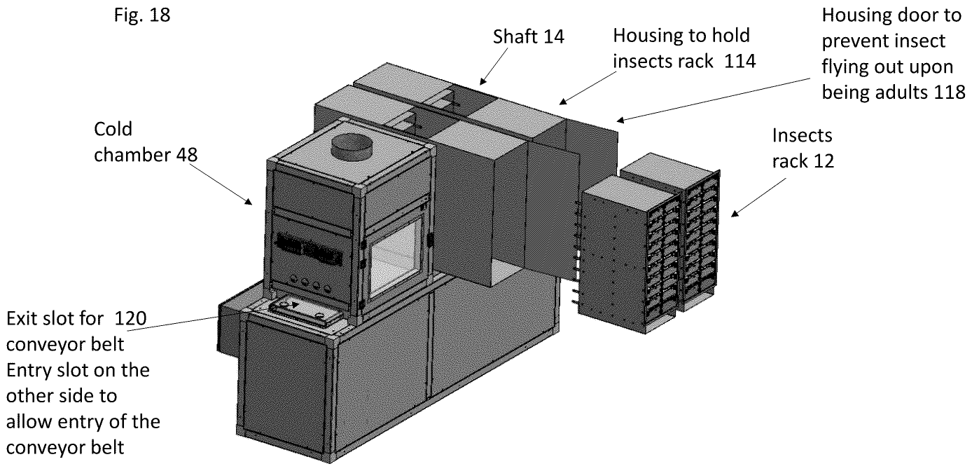

FIG. 18 illustrates insect racks 12 for holding water trays in vertical stacks being inserted into a housing 114. The housing has housing doors 118, in addition to the rack doors 110, which may be closed to enclose the water tray within so that insects can only exit towards shaft 14. Cold chamber 48, which provides cold air to immobilize the insects around the imaging area, has entry and exit 120 slots for the conveyor belt.

The terms “comprises”, “comprising”, “includes”, “including”, “having” and their conjugates mean “including but not limited to”.

The term “consisting of” means “including and limited to”.

As used herein, the singular form “a”, “an” and “the” include plural references unless the context clearly dictates otherwise.

It is appreciated that certain features of the invention, which are, for clarity, described in the context of separate embodiments, may also be provided in combination in a single embodiment and the present description is to be construed as if such embodiments are explicitly set forth herein. Conversely, various features of the invention, which are, for brevity, described in the context of a single embodiment, may also be provided separately or in any suitable subcombination or may be suitable as a modification for any other described embodiment of the invention and the present description is to be construed as if such separate embodiments, subcombinations and modified embodiments are explicitly set forth herein. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A device for rearing and sorting insects comprising:
a shaft;
a plurality of storage compartments, each compartment configured with a first closable opening for insertion of a water tray to hold pupae, and a second opening into said shaft, the inserted water tray being enclosed by said respective compartment apart from said openings;
a conveyor mechanism aligned with an exit from said shaft to catch insects emerging from said pupae via said shaft;
a classifier configured to classify insects on said conveyor mechanism into at least two classes; and
a sorter for sorting insects on said conveyor based on output of said classifier to provide a sorted group of one of said classes.

2. The device of claim 1, further comprising an air pressure source for moving air in said shaft to blow or suck said insects towards said conveyor.

3. The device of claim 2, wherein said air pressure source is located below said conveyor, said conveyor having a surface being porous and said air pressure source being configured to suck insects down said shaft towards said conveyor surface and to immobilize said insects on said surface.

4. The device of claim 2, wherein said air pressure source is located at a top of said shaft to blow insects down said shaft onto said conveyor.

5. The device of claim 1, wherein said storage compartments are in a stack and said shaft extends upwardly for a height of said stack.

6. The device of claim 1, comprising an air pressure source located to blow insects in respective storage compartments out of a respective second opening.

7. The device of claim 1, wherein inner surfaces of said storage compartments are colored not to be black or brightly colored, thereby to discourage insects from landing thereon, and/or wherein light is provided in said shaft to encourage said insects to fly into said shaft.

8. The device of claim 1, wherein the sorter comprises at least one array of suction elements across a width of said conveyor.

9. The device of claim 8, wherein said classifier is configured to record a position across said width of an insect being classified, the device being configured to activate a suction element of said array, which suction element is at said recorded width position, if said insect being classified is classified for removal.

10. The device of claim 9, configured to measure a movement of said conveyor and to carry out said activation when said insect being classified reaches said array.

11. The device of claim 10, comprising a second array of suction elements for sucking up any insect not removed by said first array.

12. The device of claim 1, further comprising a cold chamber, the cold chamber connected to receive chilled air, the chilled air being at a temperature to immobilize said insects, the cold chamber being placed around said conveyor in association with cameras, thereby to image said insects in an immobilized state for said classifier.

13. The device of claim 1, wherein said shaft is connected via a pipe to said conveyor.

14. The device of claim 1, wherein the shaft is directly over said conveyor and said storage compartments are stacked on either side of said shaft, said shaft extending at least over a height of said storage compartments.

15. The device of claim 14, comprising an array of at least two stacks of storage compartments on each side of said shaft.

16. The device of claim 14, comprising air pressure adjustment openings within said stacks.

17. The device of claim 1, wherein the stacks are removable.

18. The device of claim 1, including an outlet for the desired class of insect, the outlet being configured for attachment of a removable storage element for filling with the desired class of insect for later distribution.

19. A method for mass rearing and sorting of insects, comprising:
placing pupae in water trays;
placing said water trays in storage compartments in a stack opening into a shaft;
closing said storage compartment on all sides except into said shaft so that insects emerging from said pupae fly into said shaft;
providing air motion in said shaft to encourage said insects to fly towards a conveying mechanism;
classifying said insects on said conveying mechanism into at least two classes; and
sorting insects on said conveying mechanism based on output of said classifying to provide a sorted group of one of said classes.

20. A device for rearing and sorting insects comprising:
a shaft;
a plurality of storage compartments, each compartment configured with a first closable opening for insertion of a water tray to hold pupae, and a second opening into said shaft, the inserted water tray being enclosed by said respective compartment apart from said openings;
a conveyor belt aligned with an exit from said shaft to catch insects emerging from said pupae via said shaft; and
a classifier configured to classify objects on said conveyor belt into wanted insects and to provide a count of said wanted insects.
